(19) United States
(12) Patent Application Publication
(10) Pub. No.: US 2008/0086123 A1
(43) Pub. Date: Apr. 10, 2008

(54) Radiolucent orthopedic apparatus

(76) Inventor: Yechiel Gotfried, Kiryat Motzkin (IL)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/973,609

(22) Filed: Oct. 9, 2007

Related U.S. Application Data
(60) Provisional application No. 60/850,527, filed on Oct. 10, 2006.

(51) Int. Cl. A61B 17/00; A61B 17/58

(52) U.S. Cl. 606/59; 606/54; 606/62

(57) ABSTRACT

Apparatus and method for treating a bone of a body of a patient includes a first member, at least a portion of which is radiolucent, arranged to be attached to the bone, an attachment member for attaching the first member to the bone, and a second member, at least a portion of which is radiolucent, arranged at least partly outside of the body of the patient and coupled to the first member. The second member guides the attachment member to facilitate attachment of the first member to the bone via the attachment member. The partially radiolucent composition of the first and second members facilitates attachment of the first member to the bone while imaging the apparatus, i.e., subjecting the apparatus and bone to X-ray imaging.

### CROSS-REFERENCE TO RELATED APPLICATION

This application claims priority of U.S. provisional patent application Ser. No. 60/850,527 filed Oct. 10, 2006, the entire contents of which are incorporated by reference herein.

### FIELD OF THE INVENTION

The present invention relates generally to an orthopedic apparatus for use in treating a bone in a body of a patient and a method for treating the bone using the apparatus.

The present relates more specifically to an orthopedic apparatus for use in attaching a member to a bone while imaging the apparatus and a method for attaching a member to the bone while imaging the apparatus.

### BACKGROUND OF THE INVENTION

Biocompatible carbon fiber-reinforced thermoplastic materials are being introduced for use as implantable medical devices for orthopedic applications. Since these devices are based on a non-metallic biomaterial for medical implants, surgical procedures related thereto enable radiolucent imaging capabilities. More specifically, these devices enable a clear visualization through imaging techniques, such as X-ray and Computer Tomography (CT), in cases where X-ray visibility is advantageous. These devices can also be tailored and optimized for imaging use by adding a non-radiolucent, i.e., radio-opaque, component in variable concentrations to vary the level of X-ray contrast.

### SUMMARY OF THE INVENTION

An apparatus for treating a bone of a body of a patient in accordance with the invention includes a first member, at least a portion of which is radiolucent, arranged to be attached to the bone, one or more attachment members for attaching the first member to the bone, and a second member, at least a portion of which is radiolucent, arranged at least partly outside of the body of the patient and coupled to the first member. The second member is arranged to guide the attachment member(s) to facilitate attachment of the first member to the bone via the attachment member(s). The partially radiolucent composition of the first and second members facilitates attachment of the first member to the bone while imaging the apparatus, i.e., subjecting the apparatus and bone to X-ray imaging.

Substantially all of the first member may be radiolucent and/or substantially all of the second member may be radiolucent.

In one embodiment, the first member is arranged partly or entirely within the bone, when attached to the bone. Alternatively or additionally, the first member may be arranged partly or entirely outside of the bone, when attached to the bone. In any event, at least a part of the first member is usually engaged directly with the bone and is designed to be attached thereto in order to treat the bone, e.g., aid in healing of a fracture and the like

The first member may include a non-radiolucent portion, which facilitates X-ray identification of a disposition, location and/or orientation of the first member and/or for reinforcement parts of the first member.

The second member may include a non-radiolucent portion, which facilitates X-ray identification of a disposition, location and/or orientation of the second member and/or for reinforcement parts of the second member.

The second member may be arranged to be non-releasably coupled to the first member, i.e., fixed thereto, or alternatively, releasably coupled to the first member.

The first member may include a plurality of sub-members. For some embodiments, at least one of the sub-members is not radiolucent, i.e., made of one or more non-radiolucent materials.

The second member may additionally or alternatively include a plurality of sub-members. For some embodiments, at least one of the sub-members is not radiolucent, i.e., made of one or more non-radiolucent materials.

The first member may be made of materials such that it is not biodegradable, i.e., made of one or more non-biodegradable materials. Alternatively, the first member may be made of one or more biodegradable materials. In either embodiment, the first member may include a plurality of sub-members, at least one of which may not be radiolucent, i.e., made of one or more non-radiolucent materials.

A method for treating a bone of a body of a patient in accordance with an embodiment of the invention includes deploying a first member inside or outside the bone but at least partly inside the body and in connection with the bone, at least a portion of the first member being radiolucent, arranging or otherwise disposing a second member at least partly outside of the body of the patient, at least a portion of the second member being radiolucent, this second member being coupled to the first member before the first member is deployed in connection with the bone, and facilitating attachment of the first member to the bone by using the second member to guide one or more attachment members which attach the first member to the bone. This latter step may be performed when imaging at least the first member and the bone, i.e., while X-raying the first member to visualize the radiolucent portion(s) of the first member in order to determine when it is in an appropriate position for attachment or securing of the first member to the bone. Substantially all of the first member may be radiolucent. The same variations to the first and second members and attachment members, and other features of the apparatus described above, may be applied in the method as well.

### BRIEF DESCRIPTION OF THE DRAWINGS

The following drawings are illustrative of embodiments of the inventions disclosed herein and are not meant to limit the scope of the invention as encompassed by the claims.

FIG. 1 is a front view of a first embodiment of an apparatus for treating a bone in a body of a patient in accordance with the invention.

FIG. 2 is a front view of a second embodiment of an apparatus for treating a bone in a body of a patient in accordance with the invention.

FIG. 3 is a front view of a third embodiment of an apparatus for treating a bone in a body of a patient in accordance with the invention. FIG. 4 is a cross-sectional view taken along the line 4-4 in FIG. 3.

### DETAILED DESCRIPTION OF THE INVENTION

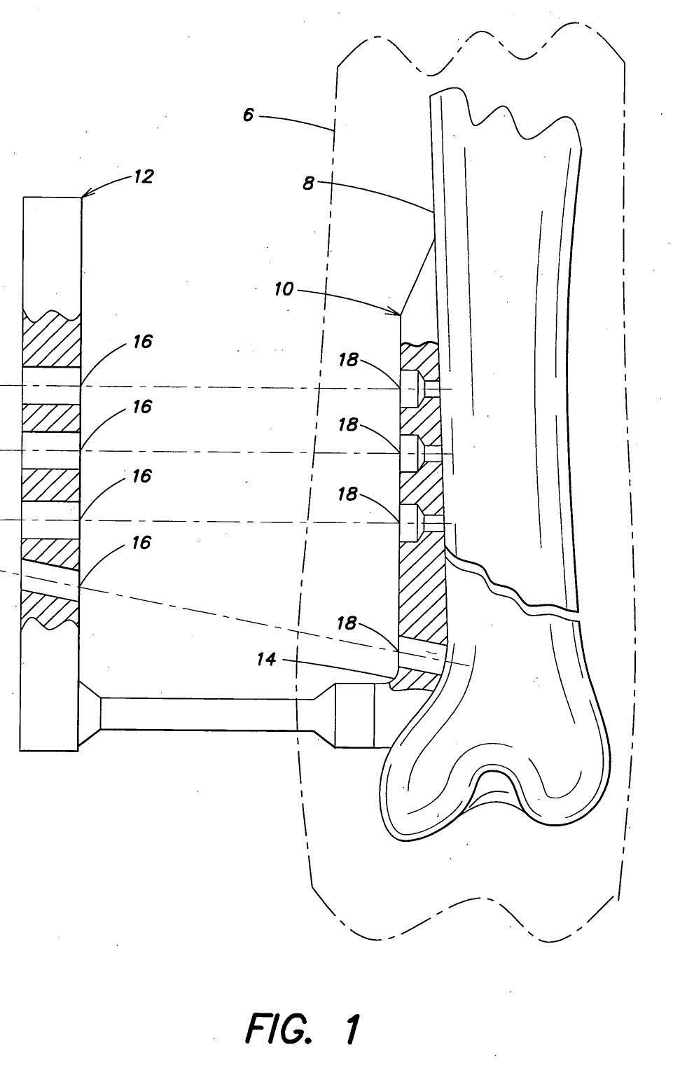

Referring to the accompanying drawings wherein like reference numerals refer to the same or similar elements, FIG. 1 is a front view of an embodiment of an apparatus for treating a bone in a body of a patient in accordance-with the present invention, which includes a first, at least partly radiolucent member 10 installed directly on a bone 8 in the patient's body 6, i.e., outside of and alongside a distal femur, and releasably coupled or non-releasably coupled (fixed) at a junction 14 to a second, at least partly radiolucent member 12 located at least partly outside the patient's body 6. The first and second members 10, 12 are thus coupled directly to each other.

A “radiolucent” material, in the context of the present patent application and in the claims, is a material which allows X-rays to pass therethrough (in varying degrees depending on the material properties of the material and the intensity of the X-rays).

The second member 12 is arranged to guide attachment members, e.g., screws (not shown in this embodiment), to facilitate attachment of the first member 10 to the bone 8. One way the second member 12 performs this function is by virtue of the presence of apertures 16 which align with apertures 18 in the first member 10 so that placement of an attachment member such as a screw through an aperture 16 in the second member 12 directs the screw into the aligning aperture 18 in the first member 10.

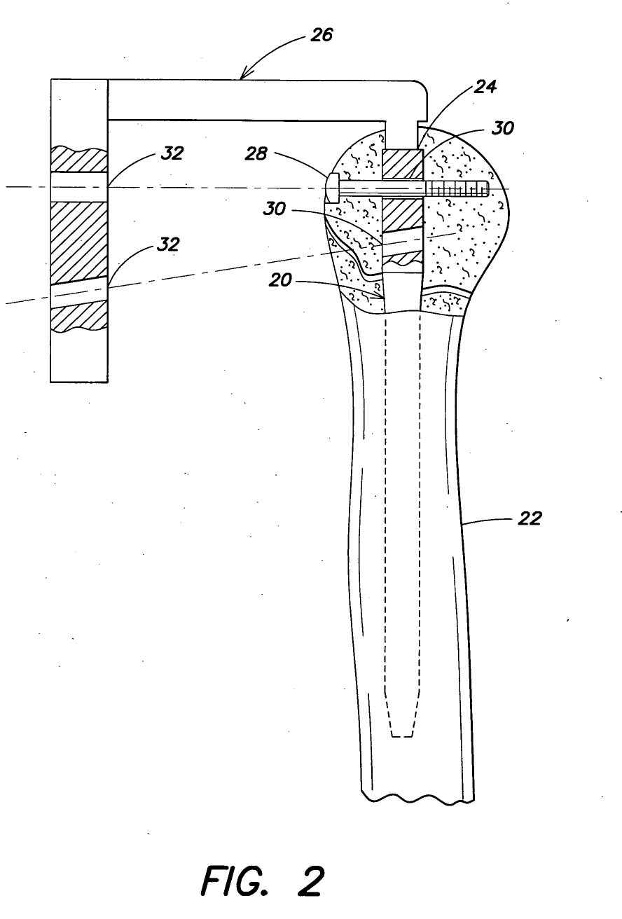

FIG. 2 is a front view of another embodiment of an apparatus for treating a bone in a patient's body in accordance with the present invention which includes a first, at least partly radiolucent member 20 installed inside the bone 22, i.e., inside a proximal humerus in this case, and releasably coupled or non-releasably coupled (fixed) at a junction 24 to a second, at least partly radiolucent member 26, located at least partly outside of the patient's body. An attachment member 28 is guided by the second member 26, via appropriate guide means as described above, i.e., aligning apertures 30, 32, to a position with respect to the first member 20 whereby attachment member 28 attaches the first member 20 to the humerus 22. Attachment member 28 is, for example, a screw which passes through an aperture 30 in the first member 20 and into bone on either side of the aperture 30. The attachment member 28 is directed into the aperture 30 in the first member 20 through an aligning aperture 32 in the second member 26.

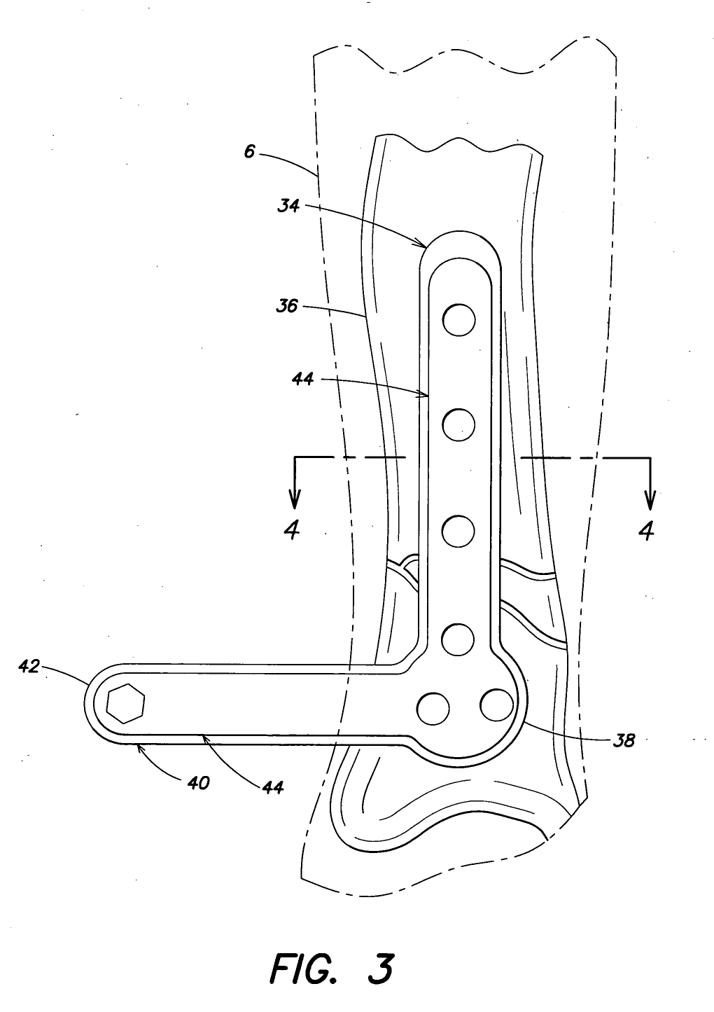

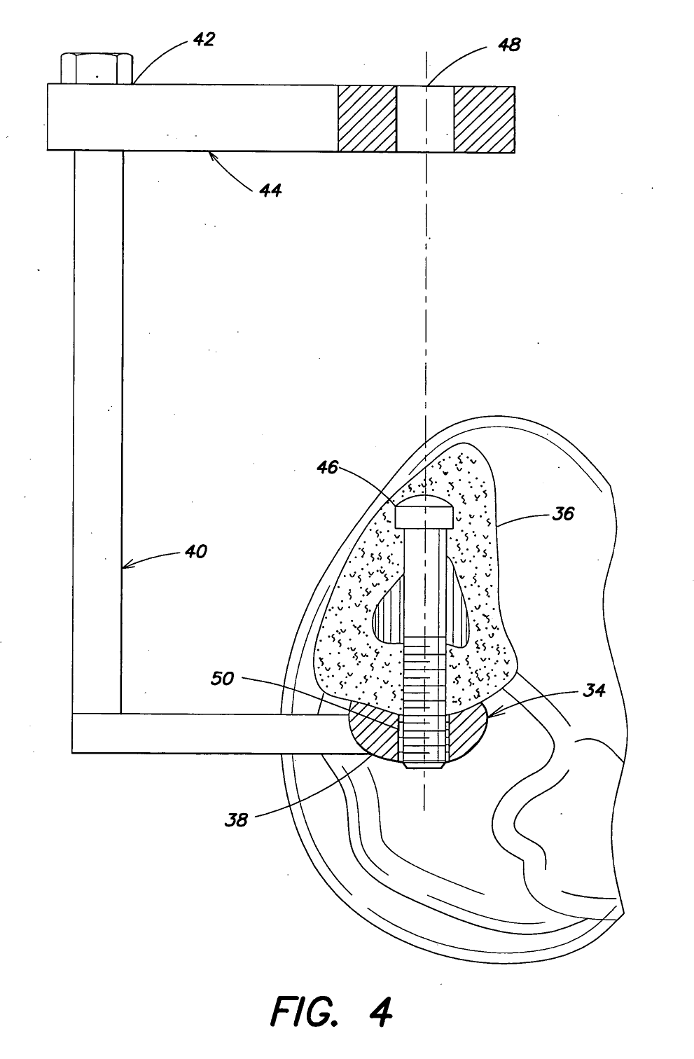

FIG. 3 is a front view of another embodiment of an apparatus for treating a bone in a patient's body 6 in accordance with the present invention which includes a first, at least partly radiolucent member 34 installed on a bone 36, i.e., in connection with a distal tibia in this case, and releasably coupled or non-releasably coupled (fixed) at a junction 38 to a second, at least partly radiolucent member 40, located at least partly outside of the patient's body 6. Second member 40 is further connected at a junction 42 to a third, at least partly radiolucent member 44.

FIG. 4 is a cross-sectional view of the apparatus shown in FIG. 3 showing an attachment member 46 which is guided by the third member 44, i.e., via an aperture 48 therein, to a position with respect to the first member 34 so that it attaches the first member 34 to the distal tibia 36. Attachment member 46 is, for example, a screw which passes through an aperture in the distal tibia 36 and then into an aperture 50 in the first member 34. The attachment member 46 is directed into the aperture 50 in the first member 34 through an aligning aperture 48 in the third member 44.

Members 10, 12, 20, 26, 34, 40 and 44 are partly radiolucent or radiolucent at least in part. Thus, each member 10, 12, 20, 26, 34, 40, 44 may be entirely radiolucent (100% radiolucent), or alternatively, each member may be greater than 15% radiolucent, greater than 30% radiolucent, greater than 60% radiolucent, or greater than 90% radiolucent by volume of the member.

In one particular embodiment, one or more non-radiolucent materials, i.e., radio-opaque materials, are incorporated into one or more of the members 10, 12, 20, 26, 34, 40, 44 in order to facilitate x-ray identification of the disposition, position, orientation and/or alignment of the members, or for reinforcing one or more of the members. In the latter case, the radio-opaque material is selected to provide a reinforcing effect to the radiolucent material.

In any of the embodiments above, each member 10, 12, 20, 26, 34, 40, 44 may comprise sub-members, at one of which is made of a non-radiolucent material. The first, second and/or third members 10, 12, 20, 26, 34, 40, 44, when including non-radiolucent materials, can be tailored and optimized for imaging use by adding the non-radiolucent members in variable concentrations to vary the level of X-ray contrast.

Members 10, 12, 20, 26, 34, 40, 44 may comprise carbon fiber composite, or another structurally-suitable radiolucent material.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description. Thus, any of the features of the different embodiments may be used in any other of the disclosed embodiments.

1. Apparatus for treating a bone of a body of a patient, comprising:
a first, at least partly radiolucent member arranged to be attachable to the bone;
an attachment member for attaching said first member to the bone; and
a second, at least partly radiolucent member arranged at least partly outside of the body and coupled to the first member, said second member being arranged to guide said attachment member to facilitate attachment of said first member to the bone via said attachment member.

2. The apparatus of claim 1, wherein said first member is entirely radiolucent.

3. The apparatus of claim 1, wherein said second member is entirely radiolucent.

4. The apparatus of claim 1, wherein said first member is structured and arranged to be situated within the bone when attached thereto.

5. The apparatus of claim 1, wherein said first member is structured and arranged to be situated outside of the bone when attached thereto.

6. The apparatus of claim 1, wherein said first member comprises a non-radiolucent portion which enables determination of a location or orientation of said first member upon exposing said first member to X-ray imaging.

7. The apparatus of claim 1, wherein said second member comprises a non-radiolucent portion which enables determination of a location or orientation of said first member upon exposing said first member to X-ray imaging.

8. The apparatus of claim 1, wherein said second member is fixed directly to said first member.

9. The apparatus of claim 1, wherein said second member is releasably coupled directly to said first member.

10. The apparatus of claim 1, wherein said first member comprises a plurality of sub-members.

11. The apparatus of claim 10, wherein at least one of said sub-members includes a non-radiolucent material.

12. The apparatus of claim 1, wherein said second member comprises a plurality of sub-members.

13. The apparatus of claim 12, wherein at least one of said sub-members includes a non-radiolucent material.

14. The apparatus of claim 1, wherein said first member is made of one or more non-biodegradable materials.

15. The apparatus of claim 1, wherein said first member is made of one or more biodegradable materials.

16. The apparatus of claim 15, wherein said first member comprises a plurality of sub-members.

17. The apparatus of claim 16, wherein at least one of said sub-members includes a non-radiolucent material.

18. The apparatus of claim 1, wherein said second member includes at least one aperture each aligning with a respective aperture on said first member and through which said attachment member passes in order to guide said attachment member into said aperture in said first member.

19. A method for treating a bone of a body of a patient, comprising:
coupling a first, at least partly radiolucent member to a second member, at least partly radiolucent member; then
deploying the first member into connection with the bone in the patient's body;
positioning the second member such that at least a part thereof is outside of the patient's body;
guiding an attachment member into engagement with the first member via the second member; and
then securing the first member to the bone via the attachment member.

20. The method of claim 19, wherein the step of coupling the second member to the first member comprises releasably fastening the second member to the first member.

21. The method of claim 19, wherein the step of coupling the second member to the first member comprises fixing the second member to the first member.

22. The method of claim 1, wherein the step of guiding the attachment member into engagement with the first member via the second member comprises passing the attachment member through an aperture in the second member which aligns with an aperture in the first member.
